(19) United States
(12) Patent Application Publication
(10) Pub. No.: US 2023/0310256 A1
(43) Pub. Date: Oct. 5, 2023

(54) COMPRESSION DEVICE AND METHOD OF USE

(71) Applicant: REATHLETE LLC, Tallahassee, FL (US)

(72) Inventor: Ziv LEVY, Moshev Ora (IL)

(21) Appl. No.: 17/701,850

(22) Filed: Mar. 23, 2022

(51) Int. Cl. A61H 9/00; A61F 7/02

(52) U.S. Cl. CPC A61H 9/0092; A61F 7/02; A61F 2007/0043; A61H 2201/0207; A61H 2201/0214

(57) ABSTRACT

A compression device wearable by a user can apply compression, heat and cold on a left lower limb and a right lower limb of the user. The compression device includes a left massager sleeve, a right massager sleeve, a computing device, a left air pressure hose, and a right air pressure hose. The left massager sleeve and the right massager sleeve surround and secure the left lower limb, and the right lower limb respectively. The computing device generates air pressure. The computing device includes a control panel to control the flow of the air pressure in the left massager sleeve and the right massager sleeve. The left air pressure hose is connected to the left massager sleeve to apply the air pressure to the left lower limb. The right air pressure hose is connected to the right massager sleeve to apply the air pressure to the right lower limb.

### FIELD OF THE INVENTION

The present invention relates to a compression device, and more particularly to a portable compression device wearable by a user for applying compression, heat and cold on a left lower limb and a right lower limb of the user.

### BACKGROUND

Typically, compression devices or clothes for applying compression to a selected area of a user’s anatomy are used in many situations. For example, compression devices are used to treat venous insufficiency or edema, or to recover muscle or to provide improved blood flow, or to prevent deep vein thrombosis (DVT). Such a compression device according to the preamble is also known from the U.S. Pat. U.S. Pat. US10772790B2 issued to Wild, et al.

Wild, et al. further describe a compression device for the limb of a mobile patient. The device includes an inflatable sleeve adapted to surround the limb; a conduit attached to the sleeve for delivering fluid to the sleeve; and a portable, wearable controller attached to the conduit that generates and controls the flow of fluid in the device.

Also known is an apparatus for providing compression and vibration therapy from U.S. Pat. US20210275390A1 filed by Hsueh; Yu-Chi; et al. Hsueh; Yu-Chi; et al. discloses an apparatus for providing compression and vibration therapy to a body part can include a housing, a pump positioned in the housing, the pump configured to generate air pressure, a compression sleeve configured to at least partially surround the body part, the compression sleeve can include an inflatable chamber in fluid communication with the pump and can be configured to apply pressure to the body part in response to receiving air from the pump, and a vibrating unit configured to apply vibration to the body part.

U.S. Pat. US20210275386A1 filed by Ravikumar; Sundaram; et al. describes a therapeutic compression apparatus for providing pressure to a limb or other body part including a bladder assembly having at least one bladder within a primary wrap and/or a secondary wrap. The primary wrap includes an inflation port to connect to an inflation means which provides either constant or varying pressure within the bladder and a check valve is included in the inflation port and/or inflation means.

These known devices do not have hot and cold features and various compression modes. Further, the existing compression devices have various disadvantages such as the sleeves being inflatable and not user-friendly. The sleeves are wrapped around the limb and the bladder are inflated by a controller that resides separately from the user. If the user must move, the sleeve must be removed. In addition, while the sleeve is on the user, the tubes connecting the bladder and controller may become entangled with the user’s limbs.

The present invention aims to resolve at least some of the problems and disadvantages mentioned above. The aim of the invention is to provide a compression device and method that eliminates those disadvantages. The present invention targets at solving at least one of the aforementioned disadvantages.

The invention thereto aims to provide a compression device wearable by a user for applying compression, heat and cold on a left lower limb and a right lower limb of the user.

There remains a need in the art for an improved and portable compression device.

Accordingly, a need arises for a portable and air compression massager and heat and cold therapy device to alleviate pain and accelerate recovery.

### SUMMARY OF THE INVENTION

The present invention and embodiments thereof serve to provide a solution to one or more of the above-mentioned disadvantages. To this end, the present invention relates to a compression device wearable by a user for applying compression, heat and cold on a left lower limb and a right lower limb of the user.

According to an embodiment herein, the compression device includes a left massager sleeve, a right massager sleeve, a computing device, a left air pressure hose, and a right air pressure hose. The left massager sleeve is adapted to surround the left lower limb. The left massager sleeve includes a first plurality of hook and loop fasteners to secure the left foot and left calf of the user. The left massager sleeve includes a left heat pad to apply heat to the left lower limb. The left massager sleeve further includes one or more left cooling pads to apply cold to the left lower limb. The right massager sleeve is adapted to surround the right lower limb. The right massager sleeve includes a second plurality of hook and loop fasteners to secure the right foot and right calf of the user. The right massager sleeve includes a right heat pad to apply heat to the right knee. The right massager sleeve further includes one or more left cooling pads to apply cold to the right lower limb. The computing device generates air pressure. The computing device includes a control panel to control the flow of the air pressure in the left massager sleeve and the right massager sleeve. The left air pressure hose supplies air pressure to the left massager sleeve in response to receiving air pressure from the computing device. The left air pressure hose is operably connected to the left massager sleeve to apply the air pressure to the left lower limb. The right air pressure hose supplies air pressure to the right massager sleeve in response to receiving air pressure from the computing device. The right air pressure hose is operably connected to the right massager sleeve to apply the air pressure to the right lower limb.

In another embodiment, the computing device includes a display screen to display a timer, a compression intensity, a heat intensity, and a plurality of massage modes.

In another embodiment, the computing device includes an intensity button to start and stop an operation of the computing device and to adjust one or more air pressure levels upon receiving a press gesture from the user.

In an embodiment, the computing device comprises a timer button to set the timer of usage to the compression device upon receiving a press gesture from the user.

In an embodiment, the computing device comprises an intensity button to start and stop an operation of the computing device and to adjust one or more air pressure levels upon receiving a press gesture from the user.

In another embodiment, the computing device includes a first massage mode button to initiate a first command pertaining to simultaneously massage the foot and the calf of the user upon receiving a press gesture from the user.

In another embodiment, the computing device includes a second massage mode button to initiate a second command pertaining to massage the foot of the user upon receiving a press gesture from the user.

In another embodiment, the computing device includes a third massage mode button to initiate a third command pertaining to massage the calf of the user upon receiving a press gesture from the user.

In another embodiment, the computing device includes a heat mode button to initiate a fourth command pertaining to adjusting one or more heating levels.

In another embodiment, each of the left massager sleeve and the right massager sleeve comprises one or more pockets to insert pre-cooled gel pads, and wherein the one or more pockets are placed inside the left massager sleeve and the right massager sleeve.

In another embodiment, the computing device includes a memory to store a plurality of instructions pertaining to the one or more air pressure levels, the first command, the second command, the third command, the fourth command, and the one or more heating levels; and a processor to execute the plurality of instructions stored in the memory.

In another embodiment, the left air pressure hose, and the right air pressure hose are inserted into the computing device to supply the air pressure.

In another embodiment, the compression device includes an AC adapter and an extension cord to power the computing device.

In another embodiment, the compression device is placed in a carry bag when not in use to provide portability.

In a second aspect, the present invention relates to a method for applying compression, heat and cold on a left lower limb and a right lower limb of the user. The method includes a step of generating air pressure using a computing device. The method includes a step of controlling the air pressure through a control panel of the computing device. The method includes a step of supplying air pressure using a left air pressure hose to a left massager sleeve and using a right air pressure hose to a right massager sleeve in response to receiving air pressure from the computing device. The method includes inserting one or more left cooling pads in one or more pockets located inside the left massager sleeve and one or more right cooling pads on one or more pockets located inside the right massager sleeve. The method includes a step of surrounding the left lower limb by a left massager sleeve and the right lower limb by a right massager sleeve and applying air pressure to the left lower limb and the right lower limb. The left massager sleeve includes a left heat pad and the right massager sleeve includes a right heat pad. The method includes a step of applying heat to the left lower limb and the right lower limb using a left heat pad and a right heat pad respectively. The method includes a step of displaying a timer, a compression intensity, a heat intensity, and a plurality of massage modes through a display screen configured within the computing device. The method includes a step of starting and stopping an operation of the computing device and adjusting one or more air pressure levels upon receiving a press gesture from the user through an intensity button present on the computing device. The method includes the step of adjusting one or more heating levels through a heat mode button present on the computing device.

In another embodiment, the one or more air pressure levels having a predefined pressure value stored in a memory of the computing device.

In another embodiment, the one or more air pressure levels comprises a first air pressure level, a second air pressure level, a third air pressure level, and a fourth air pressure level.

In another embodiment, the predefined pressure value for the first air pressure level is about 10 KPa.

In another embodiment, the predefined pressure value for the second air pressure level is about 15 KPa.

In another embodiment, the predefined pressure value for the third air pressure level is about 20 KPa.

In another embodiment, the predefined pressure value for the fourth air pressure level is about 25 KPa.

In another embodiment, the one or more heating levels having a predefined heat value stored in the memory of the computing device.

In another embodiment, the one or more heating levels comprises a first heating level, a second heating level, a third heating level, and a fourth heating level.

In another embodiment, the predefined heat value for the first heating level is about 45° C.

In another embodiment, the predefined heat value for the second heating level is about 49° C.

In another embodiment, the predefined heat value for the third heating level is about 53° C.

In another embodiment, the predefined heat value for the fourth heating level is about 56° C.

### DESCRIPTION OF FIGURES

The following description of the figures of specific embodiments of the invention is merely exemplary in nature and is not intended to limit the present teachings, their application, or their uses. Throughout the drawings, corresponding reference numerals indicate like or corresponding parts and features.

FIG. 1 shows a schematic representation of a compression device wearable by a user for applying compression on a left lower limb and a right lower limb and heat on a left knee and a right knee of the user of an embodiment of the present invention.

FIG. 2 shows a first schematic front view of the left massager sleeve and the right massager sleeve of an embodiment of the present invention.

FIG. 3 shows a schematic inner view of the left massager sleeve and the right massager sleeve of an embodiment of the present invention.

FIG. 4 shows a second schematic front view of the left massager sleeve and the right massager sleeve of an embodiment of the present invention.

FIG. 5 shows a schematic top view of the left massager sleeve and the right massager sleeve of an embodiment of the present invention.

FIG. 6 shows a first schematic side view of the right massager sleeve of an embodiment of the present invention.

FIG. 7 shows a second schematic side view of the right massager sleeve of an embodiment of the present invention.

FIG. 8 shows a schematic front-top view of the left massager sleeve and the right massager sleeve of an embodiment of the present invention.

FIG. 9 shows a schematic bottom view of the left massager sleeve and the right massager sleeve of an embodiment of the present invention.

FIG. 10 shows a first schematic side view of the left massager sleeve of an embodiment of the present invention.

FIG. 11 shows a second schematic side view of the left massager sleeve of an embodiment of the present invention.

FIG. 12 shows a schematic view of compression device of an embodiment of the present invention.

FIG. 13 shows an exploded view of the computing device of an embodiment of the present invention.

FIG. 14 shows a flowchart of a method for applying compression, heat, and cold on a left lower limb and a right lower limb of the user of an embodiment of the present invention.

### DETAILED DESCRIPTION OF THE INVENTION

The present invention concerns a compression device wearable by a user for applying compression, heat and cold on a left lower limb and a right lower limb of the user.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/-20% or less, preferably +/-10% or less, more preferably +/-5% or less, even more preferably +/-1% or less, and still more preferably +/-0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specify the presence of what follows e.g. component and do not exclude or preclude the presence of additional, non-recited components, features, element, members, steps, known in the art or disclosed therein.

Furthermore, the terms first, second, third, and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The recitation of numerical ranges by endpoints includes all numbers and fractions subsumed within that range, as well as the recited endpoints.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7, etc. of said members, and up to all said members.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures, or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention and form different embodiments, as would be understood by those in the art. For example, in the following claims, any of the claimed embodiments can be used in any combination.

In a first aspect, the invention provides/relates to a compression device wearable by a user for applying compression, heat and cold on a left lower limb and a right lower limb of the user. The compression device includes a left massager sleeve, a right massager sleeve, a computing device, a left air pressure hose, and a right air pressure hose.

The inventors have unexpectedly observed that the present invention provides a portable professional-grade air compression massager and heat and cold therapy to alleviate pain, accelerate recovery, and help the users such as an athlete, or a patient to completely relax. Further, the compression device of the present invention provides a massage that covers the calves and feet of the user.

In a preferred embodiment of the invention, the computing device includes a heat mode button to quickly relieve pain in the lower limbs.

In a particularly preferred embodiment, the computing device has various configurations to enable the user to find a massage mode that fits the user best.

In another preferred embodiment, the left massager sleeve, and the right massager sleeve include adjustable fasteners to ensure that the sleeves fit the users irrespective of their limb’s size.

However, it is obvious that the invention is not limited to this application. The method according to the invention can be applied in all sorts of wearable compression devices, massager, and heat and cold therapy devices.

The invention is further described by the following non-limiting examples which further illustrate the invention, and are not intended to, nor should they be interpreted to, limit the scope of the invention.

The present invention will be now described in more detail, referring to examples that are not limitative.

### EXAMPLES AND/OR DESCRIPTION OF FIGURES

With as a goal illustrating better the properties of the invention the following presents, as an example and limiting in no way other potential applications, a description of a number of preferred applications of the method for examining the state of the grout used in a mechanical connection based on the invention, wherein:

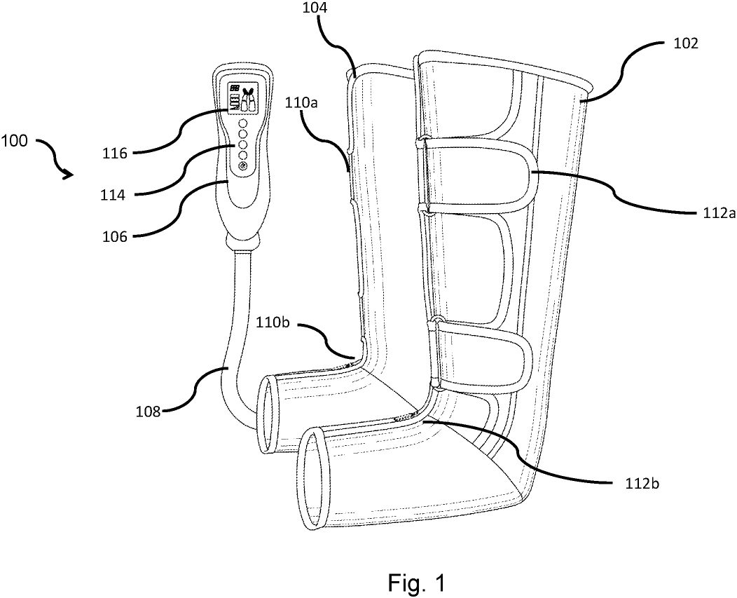

FIG. 1 shows a schematic representation of a compression device 100 wearable by a user for applying compression, heat and cold on a left lower limb and a right lower limb of the user of an embodiment of the present invention. The compression device 100 includes a left massager sleeve 102, a right massager sleeve 104, a computing device 106, and a left/right air pressure hose 108. The left massager sleeve 102 is adapted to surround the left lower limb. The right massager sleeve 104 is adapted to surround the right lower limb. The computing device 106 generates air pressure. The left air pressure hose 108 supplies air pressure to the left massager sleeve 102 in response to receiving air pressure from the computing device 106. The left air pressure hose 108 is operably connected to the left massager sleeve 102 to apply the air pressure to the left lower limb. The left massager sleeve 102 includes a first plurality of hook and loop fasteners 112a and 112b to secure the left foot and left calf of the user. The right massager sleeve 104 is adapted to surround the right lower limb. The right massager sleeve 104 includes a second plurality of hook and loop fasteners 110a and 110b to secure the right foot and right calf of the user. The first plurality of hook and loop fasteners 112a and 112b and the second plurality of hook and loop fasteners 110a and 110b are adjustable fasteners to ensure that the left massager sleeve 102, and the right massager sleeve 104 fit the users irrespective of their size. The right air pressure hose 108 is operably connected to the right massager sleeve 104 to apply the air pressure to the right lower limb. The right air pressure hose 108 supplies air pressure to the right massager sleeve 104 in response to receiving air pressure from the computing device 106. In another embodiment, the left and right air pressure hose 108 are inserted into the computing device 106 to supply the air pressure. In another embodiment, the compression device 100 includes an AC adapter and an extension cord to power the computing device 106. In another embodiment, the compression device 100 is placed in a carry bag when not in use to provide portability.

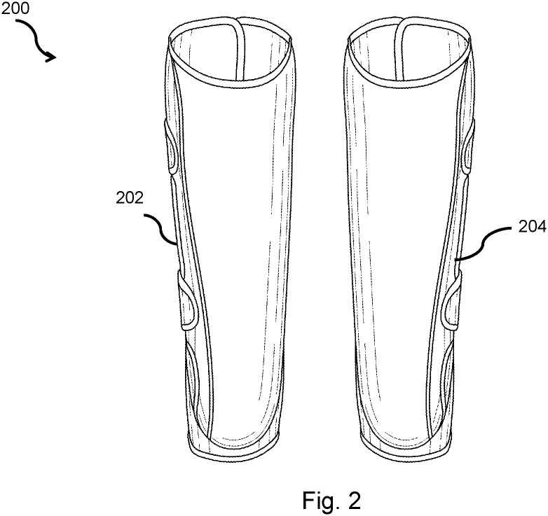

FIG. 2 shows a first schematic front view 200 of the left massager sleeve and the right massager sleeve of an embodiment of the present invention. According to an embodiment herein, the compression device includes a left massager sleeve 204 to cover the left lower limb of the user and a right massager sleeve 202 to cover the right lower limb of the user.

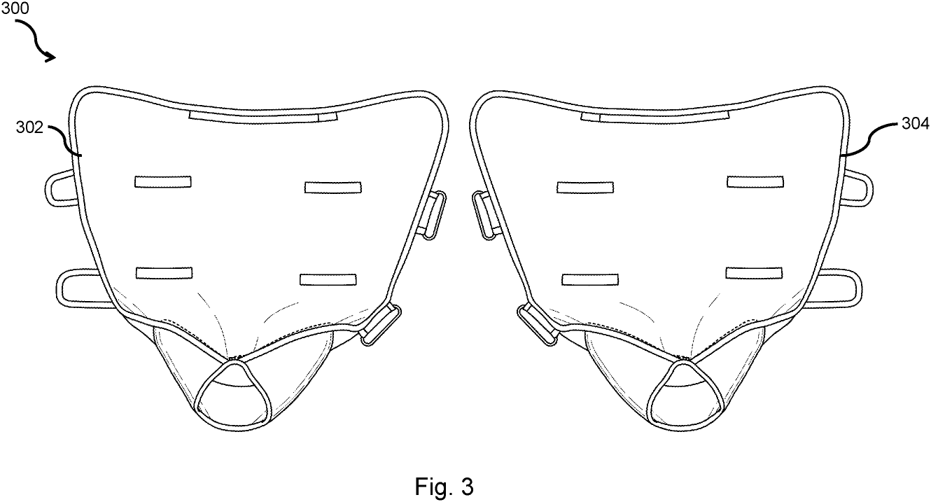

FIG. 3 shows a schematic inner view 300 of the left massager sleeve and the right massager sleeve of an embodiment of the present invention. In an embodiment, the right massager sleeve 302 and the left massager sleeve 304 act as a massager that provides compression massage to the user and reduces inflammation, and interrupts pain signals. Further, each of the right massager sleeve 302 and the left massager sleeve 304 includes cooling gel pads to apply cold to the right lower limb and the left lower limb, respectively.

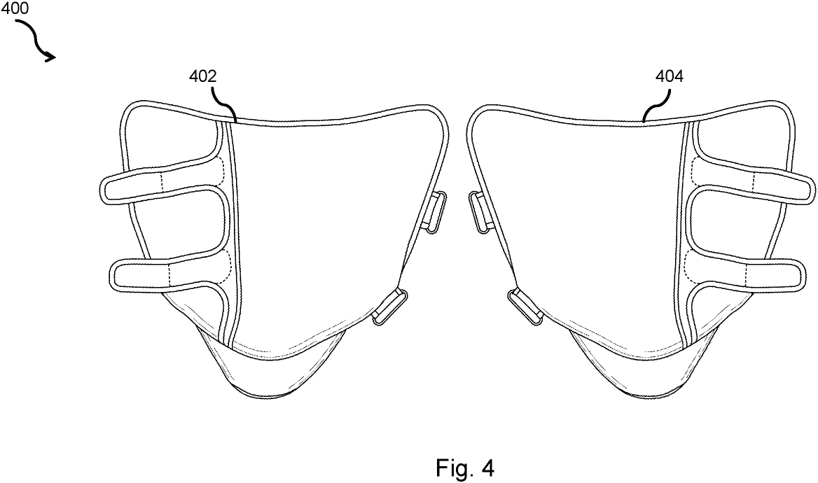

FIG. 4 shows a second schematic front view 400 of the left massager sleeve and the right massager sleeve of an embodiment of the present invention. In an embodiment, the right massager sleeve 402 and the left massager sleeve 404 provide relaxation and speed up the healing process of the user. Also, heat, cold and pressure intensify blood circulation by affecting vessels in the skin and muscles of the user.

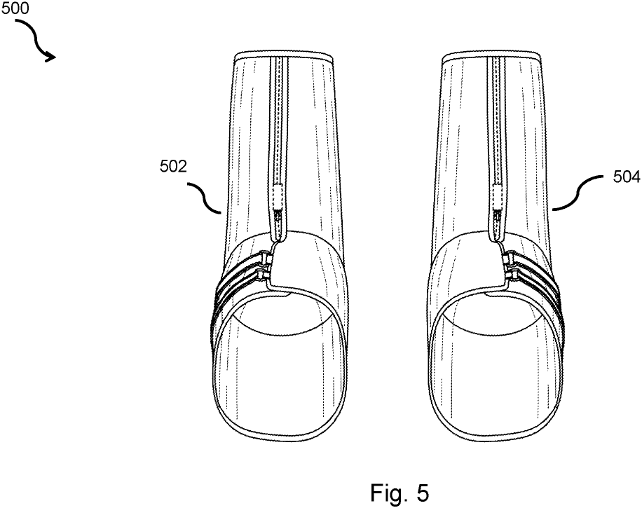

FIG. 5 shows a schematic top view 500 of the left massager sleeve and the right massager sleeve of an embodiment of the present invention. According to an embodiment herein, the left massager sleeve 502 and the right massager sleeve 504 covers the foot and calf portions of the user.

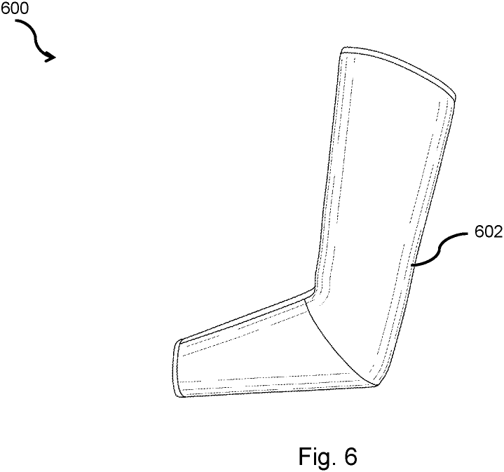

FIG. 6 shows a first schematic side view 600 of the right massager sleeve of an embodiment of the present invention. FIG. 6 illustrates left side view of the right massager sleeve 602.

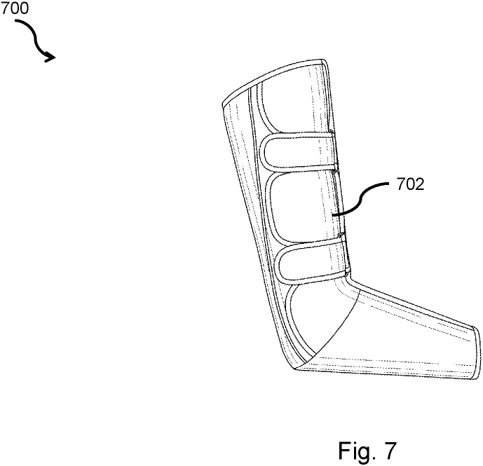

FIG. 7 shows a second schematic side view 700 of the right massager sleeve of an embodiment of the present invention. FIG. 7 illustrates right side view of the right massager sleeve 702.

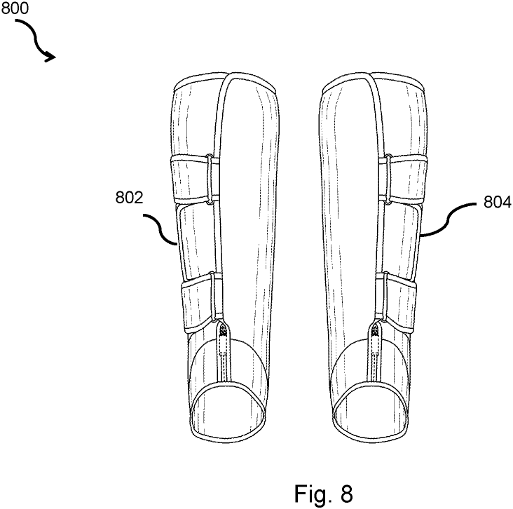

FIG. 8 shows a schematic front-top view 800 of the left massager sleeve and the right massager sleeve of an embodiment of the present invention. In an embodiment, the left massager sleeve 804 and the right massager sleeve 802 help the user to combat blood pooling, restless legs syndrome, cellulite, varicosity, trigger points, soreness, muscle fatigue, blood clots, muscle knots, and swollen veins. In an embodiment, the compression device helps the user to recover faster after workouts, and the user can relax at the end of a hard day. Further, the left massager sleeve and the right massager sleeve of the compression device provide three airbags to deliver impressive compression leading to truly therapeutic results.

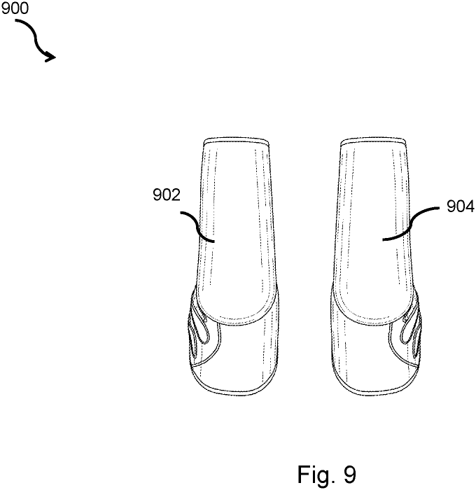

FIG. 9 shows a schematic bottom view 900 of the left massager sleeve and the right massager sleeve of an embodiment of the present invention. According to an embodiment herein, the left massager sleeve 904 covers the left feet of the user and the right massager sleeve 902 covers the right feet of the user to stimulate tissues and improve blood circulation to relieve discomfort and stiffness in the foot.

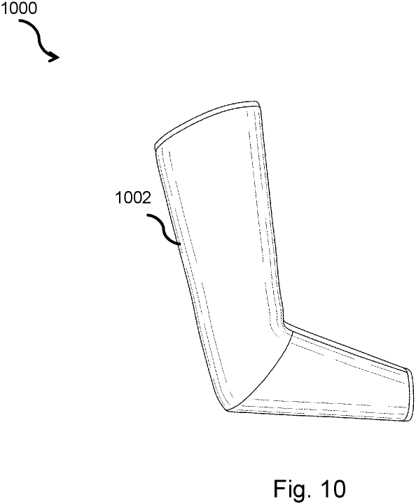

FIG. 10 shows a first schematic side view 1000 of the left massager sleeve of an embodiment of the present invention. FIG. 10 illustrates right side view of the left massager sleeve 1002.

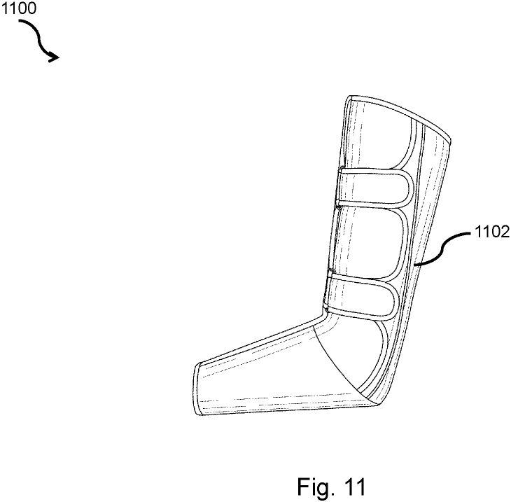

FIG. 11 shows a second schematic side view 1100 of the right massager sleeve of an embodiment of the present invention. FIG. 11 illustrates left side view of the left massager sleeve 1102.

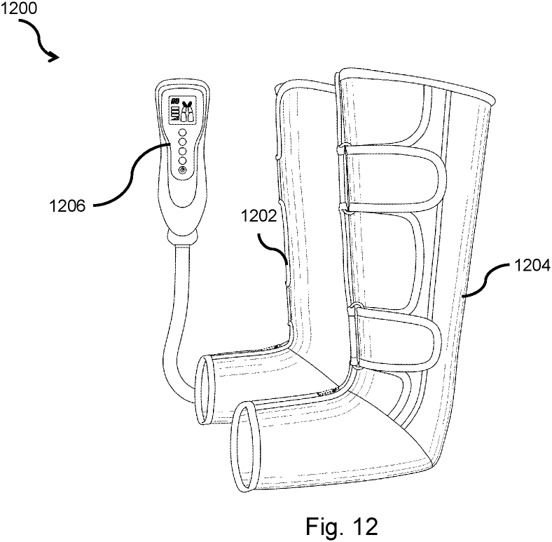

FIG. 12 shows a schematic view 1200 of compression device of an embodiment of the present invention. The compression device 1200 includes a left massager sleeve 1204, a right massager sleeve 1202, and a computing device 1206. The computing device 1206 is connected to the left massager sleeve 1204 and the right massager sleeve 1202 through the air pressure hose.

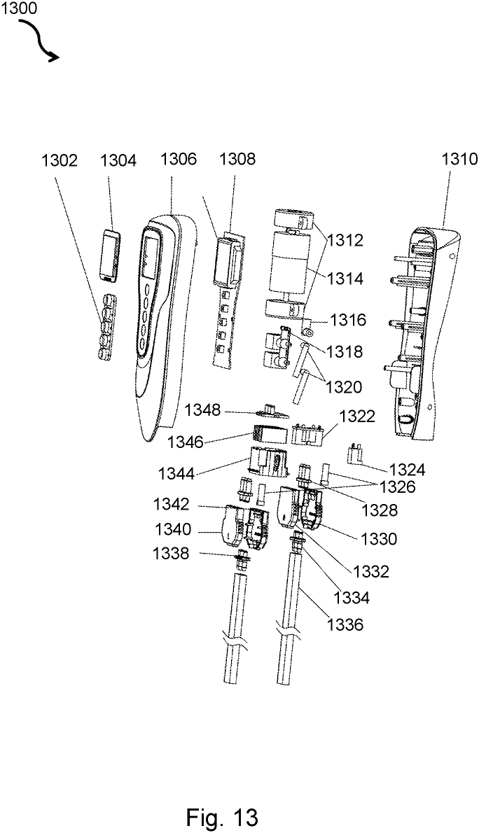

FIG. 13 shows an exploded view 1300 of the computing device of an embodiment of the present invention. FIG. 13 is explained in conjunction with FIG. 1. The computing device 106 includes a control panel 116 to control the flow of the air pressure in the left massager sleeve 102 and the right massager sleeve 104.

According to an embodiment herein, the computing device 106 includes various components such as silicone keys 1302, a mirror 1304, hand control cover 1306, hand control panel semi-finished product 1308, heat control lower cover 1310, rubber cover 1312, air pump 1314, silicone cover (air pump) 1316, triple air valve 1318, trachea 1320, DC seat semi-finished product 1322, DC seat 1324, quadruple trachea tube assembly 1326, tee 1328, R trachea transfer to the lower shell 1330, R trachea transfer to the upper shell 1332, R cross 1334, quadruple trachea 1336, L cross 1338, L trachea transfer to the top 1340, L trachea transfer lower shell 1342, hand controller 1344, silicone transfer 1346, and hand controller adaptor board 1348. In an embodiment, the computing device 106 acts as a remote controller for the compression device. In an embodiment, the silicone keys 1302 include a first massage mode button, a second massage mode button, a third massage mode button, an intensity button, and a heat mode button. According to an embodiment herein, the silicone keys 1302 are made of silicon, mirror 1304 is made of glass, the display is an LCD screen made of glass, rubber cover 1312 is made of nitrile butadiene rubber (NBR), and air pump 1314 is made of Cold rolled sheet, free-machining steel, copper wire, POM, and rubber. According to an embodiment herein, the triple air valve 1318 is made of cold-rolled sheet, free-machining steel, nylon, and glass fiber. In an embodiment, the upper cover and lower cover of the sleeves are made of Acrylonitrile butadiene styrene (ABS). In an embodiment, the computing device includes silicon elbow pipe (air valve) and air pipe that are made of silicon. In an embodiment, the computing device includes a baseboard that is made of ABS. In an embodiment, the silicone transfer 1346 is made of silicon, main transferring part, a lower cover of transferring part, a lower cover of transferring part, the lower cover of transferring part for R air pipes, and upper cover of transferring part for L air pipes are made of ABS. In an embodiment, the compression device includes an L four-way, three-way, and R four-way are made of POM. Further, DC seat 1324 and DC port are made of cold-rolled sheet and nylon plus glass fiber. Further, the four lines of air pipes of the sleeves are made of PVC. The tube parts of the hoses are made of PVC and UL2468#22AWG*2C. The thigh airbag left airbag, and the right airbag is made of TPU and Nylon 210D. The sleeve cover is made of Black 200D Nylon (polyamide) and the heating sheet is made of non-woven fabrics PET and composite fiber heating wire.

Referring to FIG. 1, the computing device 106 includes a display screen 116 to display a timer, a compression intensity, a heat intensity, and a plurality of massage modes such as a first massage mode (Mode 1), second massage mode (Mode 2), and the third massage mode (Mode 3). The massage mode can be selected by one or more massage mode buttons 114. The following table 1 depicts the working part of the body in various massage modes.

TABLE 1

| Mode | | Working part | |
|---|---|---|---|
| | | Feet | Calves |
| Mode1 | 1 | 1 | 1 |
| Mode2 | | 1 | |
| Mode3 | | | 1 |

The first massage mode button initiates a first command pertaining to simultaneously massage the foot and the calf of the user upon receiving a press gesture from the user. The second massage mode button to initiate a second command pertaining to massage the foot of the user upon receiving a press gesture from the user. The third massage mode button initiate a third command pertaining to massage the calf of the user upon receiving a press gesture from the user. The intensity button starts and stops an operation of the computing device and adjusts one or more air pressure levels upon receiving a press gesture from the user. In an embodiment, the user can press the intensity button to adjust the intensity (from Level 1 to Level 4). Further, the user can press the intensity button for at least 3 seconds to turn off the operation of the computing device. The following table 2 depicts the set pressure value for each level.

TABLE 2

The set pressure value for each level

| | |
|---|---|
| Level 1 | 10 KPa |
| Level 2 | 15 KPa |
| Level 3 | 20 KPa |
| Level 4 | 25 KPa |

Deviation: ±3KPa
There is no absolute correspondence between the air pressure value of each airbag and the time during the working process; the program only controls the air pressure value of the key control node, and the air pressure value during the inflation process is not used as a control parameter.

Further, the heat mode button initiates a fourth command pertaining to adjusting one or more heating levels such as level 1, level 2, level 3, and level 4. In an embodiment, the massage modes, compression intensity, and heat intensity are controlled independently. Furthermore, the user can press the heat mode button to cycle through various heat modes such as OFF, low, and high heat modes. For the safety of the user, the present compressor device will automatically shut down after 30 minutes of continuous use. The following table 3 depicts the preset temperature value for each heating level.

TABLE 3

The set temperature value for each level

| | |
|---|---|
| Level 1 | 45° C. |
| Level 2 | 49° C. |
| Level 3 | 53° C. |
| Level 4 | 56° C. |

Deviation: ±4° C.
The product only controls the maximum heating temperature; when the temperature is lower than the maximum temperature, the heating temperature increases with time, and the overall trend is upward.
The increase conditions of heating temperature changes with the change of heat dissipation conditions, and there is no absolute correspondence between the heating temperature and the working time during the working process of the product.

The computing device 106 includes a memory to store a plurality of instructions pertaining to the one or more air pressure levels, the first command, the second command, the third command, the fourth command, and the one or more heating levels, and control parameter. Further, the computing device includes a processor to execute the plurality of instructions or software programs stored in the memory. The instructions related to each massage mode are stored in the memory and executed by the processor as per the fowling tables 4-6 upon receiving the press gesture from the user.

TABLE 4

Mode1

| Step | Situation | Feet | Calves |
|---|---|---|---|
| 1 | P50 | C | |
| 2 | PF | C | C |
| 3 | 5 | R | R |
| 4 | 2.5 | | |
| 5 | PF | C | |
| 6 | PF | C | C |
| 7 | 5 | R | R |
| 8 | 2.5 | | |
| 9 | PF | | C |
| 10 | PF | C | C |
| 11 | 5 | R | R |
| 12 | 2.5 | | |
| 13 | PF | C | C |
| 14 | 5 | R | R |
| 15 | 8 | | |

TABLE 5

Mode2

| Step | Situation | Feet | Calves |
|---|---|---|---|
| 1 | PF | C | |
| 2 | 5 | R | |
| 3 | P90 | | |
| 4 | PF | C | |
| 5 | 5 | R | |
| 6 | P80 | | |
| 7 | PF | C | |
| 8 | 5 | R | |
| 9 | P70 | | |
| 10 | PF | C | |
| 11 | 5 | R | |
| 12 | P60 | | |
| 13 | P70 | C | |
| 14 | 5 | R | |
| 15 | P60 | | |
| 16 | P80 | C | |
| 17 | 5 | R | |
| 18 | 0.1 | | |
| 19 | P90 | C | |
| 20 | 5 | R | |
| 21 | P80 | | |
| 22 | PF | C | |
| 23 | 5 | R | |
| 24 | P90 | | |
| 25 | PF | C | |
| 26 | 5 | R | |
| 27 | 3 | | |

TABLE 6

Mode3

| Step | Situation | Feet | Calves |
|---|---|---|---|
| 1 | PF | | C |
| 2 | 5 | | R |
| 3 | P90 | | |
| 4 | PF | | C |
| 5 | 5 | | R |
| 6 | P80 | | |
| 7 | PF | | C |
| 8 | 5 | | R |
| 9 | P70 | | |
| 10 | PF | | C |
| 11 | 5 | | R |
| 12 | P60 | | |
| 13 | P70 | | C |
| 14 | 5 | | R |
| 15 | P60 | | |
| 16 | P80 | | C |
| 17 | 5 | | R |
| 18 | 0.1 | | |
| 19 | P90 | | C |
| 20 | 5 | | R |
| 21 | P80 | | |
| 22 | PF | | C |
| 23 | 5 | | R |
| 24 | P90 | | |
| 25 | PF | | C |
| 26 | 5 | | R |
| 27 | 3 | | |

In the above tables 4-6, C is indicative of “inflating”, R is indicative of “Keep pressure”, P50 is indicative of “50% of the set pressure value”, P60 is indicative of “60% of the set pressure value”, P70 is indicative of “70% of the set pressure value”, P80 is indicative of “80% of the set pressure value”, P90 is indicative of 90% of the set pressure value, PF is indicative of “The set pressure value”, and Figure is indicative of “Executing time (Unit: second, under situation column)”.

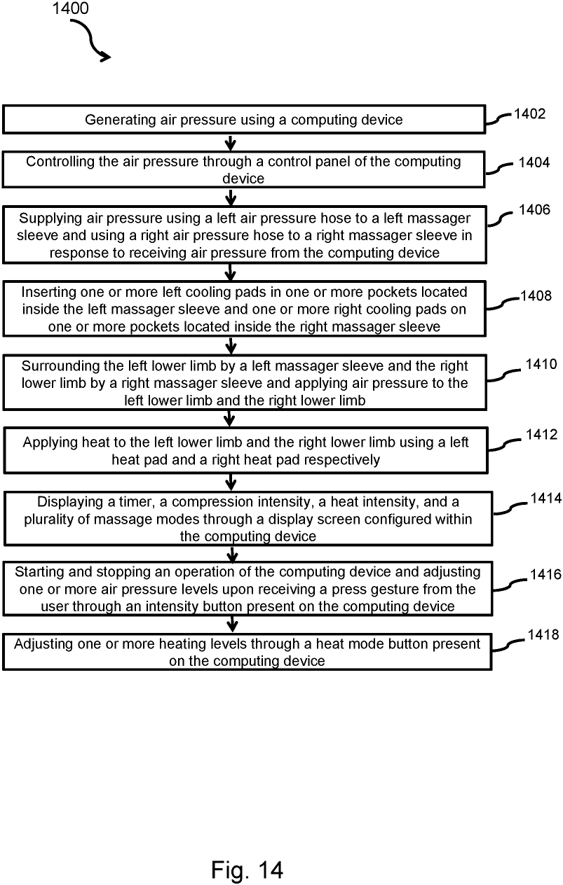

FIG. 14 shows a flowchart 1400 of a method applying compression, heat and cold on a left lower limb and a right lower limb of the user of an embodiment of the present invention. The method includes a step 1402 of generating air pressure using a computing device. The method includes a step 1404 of controlling the air pressure through a control panel of the computing device. The method includes a step 1406 of supplying air pressure using a left air pressure hose to a left massager sleeve and using a right air pressure hose to a right massager sleeve in response to receiving air pressure from the computing device. The method includes a step 1408 of inserting one or more left cooling pads in one or more pockets located inside the left massager sleeve and one or more right cooling pads on one or more pockets located inside the right massager sleeve. The method includes a step 1410 surrounding the left lower limb by a left massager sleeve and the right lower limb by a right massager sleeve and applying air pressure to the left lower limb and the right lower limb. The left massager sleeve includes a left heat pad and the right massager sleeve includes a right heat pad. The method includes a step 1412 of applying heat to the left lower limb and the right lower limb using a left heat pad and a right heat pad respectively. The method includes a step 1414 of displaying a timer, a compression intensity, a heat intensity, and a plurality of massage modes through a display screen configured within the computing device. The method includes a step 1416 of starting and stopping an operation of the computing device and adjusting one or more air pressure levels upon receiving a press gesture from the user through an intensity button present on the computing device. In another embodiment, the one or more air pressure levels having a predefined pressure value stored in a memory of the computing device. In another embodiment, the one or more air pressure levels comprises a first air pressure level, a second air pressure level, a third air pressure level, and a fourth air pressure level. In another embodiment, the predefined pressure value for the first air pressure level is about 10 KPa. In another embodiment, the predefined pressure value for the second air pressure level is about 15 KPa. In another embodiment, the predefined pressure value for the third air pressure level is about 20 KPa. In another embodiment, the predefined pressure value for the fourth air pressure level is about 25 KPa. The method includes the step 1418 of adjusting one or more heating levels through a heat mode button present on the computing device. In another embodiment, the one or more heating levels having a predefined heat value stored in the memory of the computing device. In another embodiment, the one or more heating levels comprises a first heating level, a second heating level, a third heating level, and a fourth heating level. In another embodiment, the predefined heat value for the first heating level is about 45° C. In another embodiment, the predefined heat value for the second heating level is about 49° C. In another embodiment, the predefined heat value for the third heating level is about 53° C. In another embodiment, the predefined heat value for the first heating level is about 56° C.

The present invention will now be further exemplified with reference to the following examples. The present invention is in no way limited to the given examples or to the embodiments presented in the figures.

### Example 1

A compression device shown in FIG. 1 is used to deal with tight and sore muscles. The users don’t always have a lot of time to sit and soak in a tub. The users just have to strap the sleeves on their legs and let them do the work, while the users get things done on the computer and or catch up on their TV shows. The heat setting feature helps the users from being sore so that the next day the user can go and work out at the highest level they can.

### Example 2

During an experiment, the compression device as shown in FIG. 1 is used by an athlete to apply a hot or cold pressure feature. The athlete observes that the compression device is comfortable to put on easy to take off and fits nicely around his wide calves up to his thighs. He wraps it up to his thighs and around his feet then switches the massager on from the handheld control and feels the air circulate. He found the different options for massage levels useful and he felt the intensity with each mode. It is believed that he found the fabric comfortable and breathable.

### Example 3

In the experiment of Example 2, it was found that the athlete used the compression just before bedtime and often fall asleep while it is running. He loves the fact that it automatically shuts off after 30 minutes. The heat, compressions, and different settings worked amazingly for him.

It is supposed that the present invention is not restricted to any form of realization described previously and that some modifications can be added to the presented example of fabrication without reappraisal of the appended claims. For example, the present invention has been described as referring to alleviating pain and accelerating recovery, but it is clear that the invention can be applied to heal wounds for instance or to prevent deep vein thrombosis (DVT).

It is clear that the method according to the invention, and its applications, are not limited to the presented examples.

The present invention is in no way limited to the embodiments described in the examples and/or shown in the figures. On the contrary, methods according to the present invention may be realized in many different ways without departing from the scope of the invention.

1. A compression device wearable by a user for applying compression, heat, and cold on a left lower limb and a right lower limb of the user, the compression device comprising:
a left massager sleeve adapted to surround the left lower limb, wherein the left massager sleeve comprises a first plurality of hook and loop fasteners to secure left foot and left calf of the user, wherein the left massager sleeve comprises:
a left heat pad to apply heat to the left lower limb; and
one or more left cooling gel pads to apply cold to the left lower limb;
a right massager sleeve adapted to surround the right lower limb, wherein the right massager sleeve comprises a second plurality of hook and loop fasteners to secure the right foot and right calf of the user, wherein the right massager sleeve comprises:
a right heat pad to apply heat to the right lower limb; and
one or more right cooling gel pads to apply cold to the right lower limb;
a computing device to generate air pressure, wherein the computing device comprises: a control panel to control a flow of the air pressure in the left massager sleeve and the right massager sleeve;
a left air pressure hose to supply the air pressure to the left massager sleeve in response to receiving the air pressure from the computing device, wherein the left air pressure hose is operably connected to the left massager sleeve to apply the air pressure to the left lower limb; and
a right air pressure hose to supply the air pressure to the right massager sleeve in response to receiving the air pressure from the computing device, wherein the right air pressure hose is operably connected to the right massager sleeve to apply the air pressure to the right lower limb.

2. The compression device as claimed in claim 1, wherein the computing device comprises a display screen to display a timer, a compression intensity, a heat intensity, and a plurality of massage modes.

3. The compression device as claimed in claim 1, wherein the computing device comprises a timer button to set the timer of usage to the compression device upon receiving a press gesture from the user.

4. The compression device as claimed in claim 1, wherein the computing device comprises an intensity button to start and stop an operation of the computing device and to adjust one or more air pressure levels upon receiving a press gesture from the user.

5. The compression device as claimed in claim 1, wherein the computing device comprises a first massage mode button to initiate a first command pertaining to simultaneously massage the foot and calf of the user upon receiving a press gesture from the user.

6. The compression device as claimed in claim 1, wherein the computing device comprises a second massage mode button to initiate a second command pertaining to massage the foot of the user upon receiving a press gesture from the user.

7. The compression device as claimed in claim 1, wherein the computing device comprises a third massage mode button to initiate a third command pertaining to massage the calf of the user upon receiving a press gesture from the user.

8. The compression device as claimed in claim 1, wherein the computing device comprises a heat mode button to initiate a fourth command pertaining to adjusting one or more heating levels.

9. The compression device as claimed in claim 1, wherein each of the left massager sleeve and the right massager sleeve comprises one or more pockets to insert precooled gel pads, and wherein the one or more pockets are placed inside the left massager sleeve and the right massager sleeve.

10. The compression device as claimed in claim 1, wherein the computing device comprises a memory to store a plurality of instructions pertaining to the one or more air pressure levels, a first command, a second command, a third command, a fourth command, and the one or more heating levels; and a processor to execute the plurality of instructions stored in the memory.

11. The compression device as claimed in claim 1, wherein the left air pressure hose, and the right air pressure hose are inserted into the computing device to supply the air pressure.

12. The compression device as claimed in claim 1 comprises an AC adapter and an extension cord to power the computing device.

13. The compression device as claimed in claim 1 is placed in a carry bag when not in use to provide portability.

14. A method for applying compression, heat and cold on a left lower limb and a right lower limb of the user, the method comprising:
generating air pressure using a computing device;
controlling the air pressure through a control panel of the computing device;
supplying the air pressure using a left air pressure hose to a left massager sleeve and using a right air pressure hose to a right massager sleeve in response to receiving the air pressure from the computing device;
inserting one or more left cooling pads in one or more pockets located inside the left massager sleeve and one or more right cooling pads on one or more pockets located inside the right massager sleeve;
surrounding the left lower limb by a left massager sleeve and the right lower limb by a right massager sleeve and applying the air pressure to the left lower limb and the right lower limb, wherein the left massager sleeve comprises a left heat pad and the right massager sleeve comprises a right heat pad; and
applying heat to the left lower limb and the right lower limb using a left heat pad and a right heat pad respectively.

15. The method as claimed in claim 14, further comprising a step of displaying a timer, a compression intensity, a heat intensity, and a plurality of massage modes through a display screen configured within the computing device.

16. The method as claimed in claim 14, further comprising a step of starting and stopping an operation of the computing device and adjusting one or more air pressure levels upon receiving a press gesture from the user through an intensity button present on the computing device.

17. The method as claimed in claim 16, wherein the one or more air pressure levels having a predefined pressure value stored in a memory of the computing device.

18. The method as claimed in claim 17, wherein the one or more air pressure levels comprises a first air pressure level, a second air pressure level, a third air pressure level, and a fourth air pressure level.

19. The method as claimed in claim 18, wherein the predefined pressure value for the first air pressure level is about 10 KPa.

20. The method as claimed in claim 18, wherein the predefined pressure value for the second air pressure level is about 15 KPa.

21. The method as claimed in claim 18, wherein the predefined pressure value for the third air pressure level is about 20 KPa.

22. The method as claimed in claim 18, wherein the predefined pressure value for the fourth air pressure level is about 25 KPa.

23. The method as claimed in claim 14, further comprising a step of adjusting one or more heating levels through a heat mode button present on the computing device.

24. The method as claimed in claim 23, wherein the one or more heating levels having a predefined heat value stored in the memory of the computing device.

25. The method as claimed in claim 24, wherein the one or more heating levels comprises a first heating level, a second heating level, a third heating level, and a fourth heating level.

26. The method as claimed in claim 25, wherein the predefined heat value for the first heating level is about 45° C.

27. The method as claimed in claim 25, wherein the predefined heat value for the second heating level is about 49° C.

28. The method as claimed in claim 25, wherein the predefined heat value for the third heating level is about 53° C.

29. The method as claimed in claim 25, wherein the predefined heat value for the fourth heating level is about 56° C.
